(19) United States
(12) Patent Application Publication
(10) Pub. No.: US 2008/0075710 A1
(43) Pub. Date: Mar. 27, 2008

(54) Herbal composition for treating hangovers

(75) Inventors: Erik T. Cornett, Herrin, IL (US); Gerad A. Harsy, Duquoin, IL (US)

Correspondence Address: NIXON PEABODY, LLP, 161 N. CLARK ST., 48TH FLOOR, CHICAGO, IL, 60601-3213, US

(73) Assignee: LIQUID POTIONS LLC

(21) Appl. No.: 11/900,350

(22) Filed: Sep. 11, 2007

Related U.S. Application Data
(60) Provisional application No. 60/846,933, filed on Sep. 25, 2006.

(51) Int. Cl. A61K 31/025; A61K 31/195; A61K 31/335; A61K 31/34; A61K 31/50; A61K 31/51; A61K 31/525; A61K 33/00; A61K 33/04; A61K 33/32; A61K 38/48; A61P 43/00

(52) U.S. Cl. 424/94.63; 424/641; 424/702; 424/722; 514/168; 514/249; 514/251; 514/276; 514/452; 514/474; 514/562; 514/756

(57) ABSTRACT

An herbal composition is adapted to treat hangovers. In one embodiment, the herbal composition comprises potassium, thiamin (vitamin B-1), magnesium, silymarin and N-acetyl-L-cysteine (acetylcysteine), or any pharmaceutically acceptable salts thereof. In a method of treating hangovers, an herbal composition is provided that comprises potassium, thiamin, magnesium, silymarin and N-acetyl-L-cysteine. The herbal composition is placed in the body so as to alleviate or reduce the effects of a hangover.

### CROSS-REFERENCE TO RELATED APPLICATION

This application claims priority to U.S. Provisional Application Ser. No. 60/846,933 filed on Sep. 25, 2006, which is incorporated by reference in its entirety.

### FIELD OF THE INVENTION

The present invention generally relates to an herbal composition and a method of using the same. More specifically, the present invention is directed to an herbal composition that is used to treat hangovers caused by, for example, alcoholic beverages and methods of using the same.

### BACKGROUND OF THE INVENTION

Ethyl alcohol or ethanol is often found in alcoholic beverages intended for ingestion such as beer, wine, and spirits. Consumption of ethyl alcohol may be followed by a condition referred to as a hangover. Common hangover symptoms include headache, dehydration, nausea, lethargy, congestion, diarrhea, and/or fever. These symptoms may be particularly severe, especially after heavy consumption of alcoholic beverages. While typically not life-threatening, these symptoms are unpleasant and may interfere with a person's performance at a job or at home.

The hangover is due mainly to the toxic effects of acetaldehyde, a byproduct of the breakdown of ethyl alcohol in the liver. In addition, alcoholic beverages themselves have toxins known as congeners, which are the byproducts of fermentation and distillation. After heavy alcoholic beverage consumption, the accumulated toxins in the liver disturb the normal metabolism and result in hangover symptoms. The hangover symptoms are unpleasant, and seem to vary in intensity according to the individual and the amount of alcoholic beverages consumed.

There is a need for a treatment of ethyl alcohol-induced hangover symptoms. The treatment desirably alleviates or eliminates the various symptoms of the hangover without aggravating them further. The treatments for hangover symptoms should allow the person to enjoy the desired social effects of alcohol consumption, while addressing the later negative effects of alcohol consumption.

### SUMMARY OF THE INVENTION

An herbal composition is adapted to treat hangovers. In one embodiment, the herbal composition comprises potassium, thiamin (vitamin B-1), magnesium, silymarin and N-acetyl-L-cysteine (acetylcysteine), or any pharmaceutically acceptable salts thereof. In one method of treating hangovers, an herbal composition is provided that comprises potassium, thiamin, magnesium, silymarin and N-acetyl-L-cysteine. The herbal composition is placed in the body so as to alleviate or reduce the effects of a hangover.

In another embodiment, an herbal composition is adapted to treat hangovers. The herbal composition comprises potassium, thiamin (vitamin B-1), magnesium, silymarin, folic acid, zinc and bromelain or any pharmaceutically acceptable salts thereof.

In another method, an herbal composition is provided comprising potassium, thiamin (vitamin B-1), magnesium, and silymarin or any pharmaceutically acceptable salts thereof. The herbal composition is provided in the body so as to alleviate or reduce the effects of a hangover.

### DETAILED DESCRIPTION OF THE ILLUSTRATED EMBODIMENTS

This present invention is directed to an herbal composition for the replacement of vitamins, minerals, herbs, amino acids and/or nutrients depleted by ethyl alcohol consumption. The herbal composition is also adapted to assist in decreasing the adverse symptoms of ethyl alcohol consumption (i.e., a hangover) such as, for example, detoxifying the free radicals and toxins as a result of ethyl alcohol consumption. The herbal composition may be ingested into the body. Additionally, the herbal composition is also adapted to assist in reducing nausea. The herbal composition also assists in relieving the symptoms of Wemicke's encephalopathy, which is mental confusion or delirium occurring in combination with paralysis of the eye muscles, nystagmus and an unsteady gait. Wemicke's encephalopathy is caused by a deficiency of thiamin (vitamin B1). The herbal composition also assists in relieving the symptoms of Korsakoff's syndrome, which is an organic disorder affecting the brain that results in a memory defect in which new information fails to be recalled although events from the past are still recalled. Korsakoff's syndrome is caused by a deficiency of thiamin.

A hangover (alcohol consumption) may create low blood sugar, which can lead to hypoglycemia. The herbal composition may also assist in reducing low blood sugar, which reduces the likelihood of hypoglycemia. The herbal composition may also increase the energy of an individual and, thus, reduce lethargy and malaise.

The herbal composition may be located in various dosage forms. For example, the herbal composition may be located in the following dosage forms: solution, suspension, elixir, syrup, emulsion, lozenge, tablet, capsule, pill, sublingual tablet, suppository, oral gels, topical gels, topical creams, topical ointments and topical lotions. The herbal composition is typically located in the following dosage forms: solution, tablet or capsule. It is contemplated that the herbal composition may be located in other dosage forms. The herbal composition may be placed in the body by, for example, ingesting. It is contemplated that the herbal composition may enter into the body by other methods.

According to one embodiment, an herbal composition comprises potassium, thiamin (vitamin B-1), magnesium, silymarin and N-acetyl-L-cysteine (acetylcysteine). These ingredients are commonly available and may be obtained from commercial sources. The herbal composition also may use any pharmaceutically acceptable salt of the above ingredients. Potassium is an electrolyte that is often depleted from the body due to the diuretic effect of alcohol. Thiamin (also referred to as thiamine) is a vitamin depleted from the body during alcohol consumption. Magnesium is a mineral that is required for enzymatic metabolism to properly function. Silymarin is an herbal supplement that assists in eliminating toxins from the liver. Silymarin may be available in, for example, milk thistle. N-acetyl-L-cysteine is an amino acid with antioxidant properties that assists in maintaining glutathione levels in the liver. N-acetyl-L-cysteine may also be referred to as acetylcysteine, cysteine, L-cysteine.

In one specific embodiment, an herbal composition comprises an amount of potassium being from about 25 mg to 3,000 mg, an amount of thiamin being from about 1 mg to about 300 mg, an amount of magnesium being from about 25 mg to about 1,000 mg, an amount of silymarin being from about 25 mg to about 15,000 mg, and an amount of N-acetyl-L-cysteine being from about 25 mg to 600 mg. It is desirable to have effective amounts of these ingredients in the herbal composition to treat, for example, hangovers.

In another specific embodiment using at least potassium, thiamin (vitamin B-1), magnesium, silymarin and N-acetyl-L-cysteine (acetylcysteine), the amount of potassium is from about 0.1 to about 98 wt. % of the herbal composition, the amount of thiamin is from about 0.01 to about 75 wt. % of the herbal composition, the amount of magnesium is from about 0.1 to about 95 wt. % of the herbal composition, the amount of silymarin is from about 0.5 to about 99.9 wt. % of the herbal composition, and the amount of N-acetyl-L-cysteine (acetylcysteine) is from about 0.1 to about 90 wt. % of the herbal composition.

In another specific embodiment using at least potassium, thiamin, magnesium, silymarin and N-acetyl-L-cysteine, the amount of potassium is from about 2 to about 60 wt. % of the herbal composition, the amount of thiamin is from about 0.1 to about 25 wt. % of the herbal composition, the amount of magnesium is from about 4 to about 85 wt. % of the herbal composition, the amount of silymarin is from about 3 to about 75 wt. % of the herbal composition, and the amount of N-acetyl-L-cysteine is from about 3 to about 80 wt. % of the herbal composition.

In another specific embodiment using at least potassium, thiamin, magnesium, silymarin and N-acetyl-L-cysteine, the amount of potassium is from about 5 to about 55 wt. % of the herbal composition, the amount of thiamin is from about 0.1 to about 5 wt. % of the herbal composition, the amount of magnesium is from about 5 to about 75 wt. % of the herbal composition, the amount of silymarin is from about 5 to about 75 wt. % of the herbal composition, and the amount of N-acetyl-L-cysteine is from about 5 to about 75 wt. % of the herbal composition.

According to another embodiment, the herbal composition may comprise additional ingredients besides potassium, thiamin, magnesium, silymarin and N-acetyl-L-cysteine. For example, the herbal composition may comprise potassium, thiamin, magnesium, silymarin and N-acetyl-L-cysteine and one or more of the following: ascorbic acid (vitamin C), riboflavin (vitamin B-2), proanthocyandins, vitamin E, zinc and folic acid. The herbal composition also may use any pharmaceutically acceptable salt of the above ingredients.

Ascorbic acid (vitamin C) is an antioxidant utilized in carbohydrate metabolism and cellular respiration. Riboflavin (vitamin B-2) is a vitamin required for tissue respiration and may become deficient during alcohol intake. Proanthocyandins are herbal antioxidants with greater potency than vitamin E. Proanthocyandins assist in reducing or eliminating free radicals as a result of alcohol. Proanthocyandins may be found in, for example, grape seed extract. Vitamin E is an antioxidant that assists in preserving red blood cell integrity and aids in eliminating free radicals. Zinc is necessary for the breakdown of alcohol and is often depleted during alcohol consumption. Folic acid is a vitamin that plays a major role in intracellular metabolism and reduces damage to DNA. Folic acid is also referred to as pteroylglutamic acid, pteroylmonoglutamic acid or pteroylpolyglutamate.

In another embodiment, an herbal composition comprises an amount of potassium being from about 25 mg to 3,000 mg, an amount of thiamin being from about 1 mg to about 300 mg, an amount of magnesium being from about 25 mg to about 1,000 mg, an amount of silymarin being from about 25 mg to about 15,000 mg, an amount of N-acetyl-L-cysteine being from about 25 mg to 600 mg, an amount of ascorbic acid being from about 60 mg to about 2,500 mg, an amount of riboflavin being from about 1 mg to about 1,000 mg, an amount of proanthocyandins being from about 40 mg to about 300 mg, an amount of zinc being from about 8 mg to about 400 mg, and an amount of folic acid being from about 0.2 mg to about 1 mg. It is desirable to have effective amounts of these ingredients in the herbal composition to treat, for example, hangovers.

In another specific embodiment, the amount of potassium is from about 0.1 to about 95 wt. % of the herbal composition, the amount of thiamin is from about 0.01 to about 60 wt. % of the herbal composition, the amount of magnesium is from about 0.1 to about 85 wt. % of the herbal composition, the amount of silymarin is from about 0.3 to about 99 wt. % of the herbal composition, the amount of N-acetyl-L-cysteine is from about 0.1 to about 85 wt. % of the herbal composition, the amount of ascorbic acid is from about 0.3 to about 95 wt. % of the herbal composition, the amount of riboflavin is from about 0.01 to about 85 wt. % of the herbal composition, the amount of proanthocyandins is from about 0.1 to about 65 wt. % of the herbal composition, the amount of zinc is from about 0.05 to about 70 wt. % of the herbal composition, and the amount of folic acid is from 0.001 to about 0.5 wt. % of the herbal composition.

In another specific embodiment, the amount of potassium is from about 1 to about 35 wt. % of the herbal composition, the amount of thiamin is from about 0.05 to about 10 wt. % of the herbal composition, the amount of magnesium is from about 1 to about 70 wt. % of the herbal composition, the amount of silymarin is from about 1 to about 60 wt. % of the herbal composition, the amount of N-acetyl-L-cysteine is from about 1 to about 60 wt. % of the herbal composition, the amount of ascorbic acid is from about 3 to about 90 wt. % of the herbal composition, the amount of riboflavin is from about 0.05 to about 55 wt. % of the herbal composition, the amount of proanthocyandins is from about 2 to about 65 wt. % of the herbal composition, the amount of zinc is from about 0.5 to about 50 wt. % of the herbal composition, and the amount of folic acid is from about 0.1 to about 0.5 wt. % of the herbal composition.

According to another embodiment, the herbal composition may comprise additional ingredients besides potassium, thiamin, magnesium, silymarin and N-acetyl-L-cysteine. For example, the herbal composition may comprise potassium, thiamin, magnesium, silymarin and N-acetyl-L-cysteine and one or more of the following: ascorbic acid (vitamin C), riboflavin, proanthocyandins, vitamin E, zinc, folic acid, vitamin A, pyridoxine (vitamin B-6), ginger root or ginger root extract, glutathione, bromelain, selenium and salicin. The herbal composition also may use any pharmaceutically acceptable salt of the above ingredients.

Vitamin A is an antioxidant found deficient in hepatic cirrhosis, a condition developed from continuous over-consumption of alcohol. Pyridoxine (vitamin B-6) is a vitamin involved in carbohydrate metabolism and deficiencies may occur from alcohol consumption. Ginger root or ginger root extract is an herbal supplement commonly used to relieve nausea. One non-limiting example of glutathione is L-glutathione. L-glutiathione is an amino acid commonly depleted during alcohol consumption and also needed to detoxify harmful chemicals. Glutathione is also referred to as N-(N-L-gamma-glutamyl-L-cysteinyl)glycine. One source of glutathione is whey protein such as, for example, bovine whey protein concentrate.

Bromelain is an herbal supplement used as an anti-inflammatory by reducing kininogen and bradykinin serum tissue levels and has an influence on prostaglandin synthesis. Bromelain is also referred to as ananas comosus (pineapple) and is synonymous with the following: ananas ananas, ananas duckei, ananas sativus, bromelia ananas and bromelia comosa. Selenium is an antioxidant when combined with vitamin E has a positive effect in reducing the production of prostaglandins and leukotrienes, two chemicals known to cause pain and inflammation. Salicin is an herbal supplement used as an analgesic. Salicin is also referred to as salix alba, salix daphnoides, salix fragilis, salix pentandra, salix purpurea and other salix species.

In another embodiment, an herbal composition comprises an amount of potassium being from about 25 mg to 3,000 mg, an amount of thiamin being from about 1 mg to about 300 mg, an amount of magnesium being from about 25 mg to about 1,000 mg, an amount of silymarin being from about 25 mg to about 15,000 mg, an amount of N-acetyl-L-cysteine being from about 25 mg to 600 mg, an amount of ascorbic acid being from about 60 mg to about 2,500 mg, an amount of riboflavin being from about 1 mg to about 1,000 mg, an amount of proanthocyandins being from about 40 mg to about 300 mg, an amount of zinc being from about 10 mg to about 400 mg, an amount of folic acid being from about 0.2 mg to about 1 mg, an amount of vitamin A being from about 1 mg to about 15 mg, an amount of pyridoxine being from about 1 mg to about 500 mg, an amount of ginger root being from about 5 mg to about 4,000 mg, an amount of glutathione being from about 2.5 mg to about 5,000 mg, an amount of bromelain being from about 150 mg to about 3,000 mg, an amount of selenium being from about 0.05 mg to about 0.7 mg, and an amount of salicin being from about 60 mg to about 120 mg. It is desirable to have effective amounts of these ingredients in the herbal composition to treat, for example, hangovers.

In a further specific embodiment, the amount of potassium is from about 0.1 to about 90 wt. % of the herbal composition, the amount of thiamin is from 0.001 to about 40 wt. % of the herbal composition, the amount of magnesium is from about 0.1 to about 70 wt. % of the herbal composition, the amount of silymarin is from about 0.1 to about 99 wt. % of the herbal composition, the amount of N-acetyl-L-cysteine is from about 0.1 to about 60 wt. % of the herbal composition, the amount of ascorbic acid is from about 0.2 to about 90 wt. % of the herbal composition, the amount of riboflavin is from 0.001 to about 70 wt. % of the herbal composition, the amount of proanthocyandins is from about 0.1 to about 50 wt. % of the herbal composition, the amount of zinc is from about 0.02 to about 50 wt. % of the herbal composition, the amount of folic acid is from 0.001 to about 0.3 wt. % of the herbal composition, the amount of vitamin A is from 0.0001 to about 5 wt. % of the herbal composition, the amount of pyridoxine is from 0.001 to about 60 wt. % of the herbal composition, the amount of ginger root or ginger root extract is from about 0.01 to about 90 wt. % of the herbal composition, the amount of glutathione is from about 0.01 to about 95 wt. % of the herbal composition, the amount of bromelain is from about 0.5 to about 95 wt. % of the herbal composition, the amount of selenium is from 0.0001 to about 0.2 wt. % of the herbal composition, and the amount of salicin is from about 0.15 to about 25 wt. % of the herbal composition.

In another specific embodiment, the amount of potassium is from about 1 to about 20 wt. % of the herbal composition, the amount of thiamin is from about 0.02 to about 5 wt. % of the herbal composition, the amount of magnesium is from about 10 to about 50 wt. % of the herbal composition, the amount of silymarin is from about 0.5 to about 40 wt. % of the herbal composition, the amount of N-acetyl-L-cysteine is from about 0.5 to about 40 wt. % of the herbal composition, the amount of ascorbic acid is from about 2 to about 70 wt. % of the herbal composition, the amount of riboflavin is from about 0.02 to about 40 wt. % of the herbal composition, the amount of proanthocyandins is from about 1 to about 40 wt. % of the herbal composition, the amount of zinc is from about 0.2 to about 35 wt. % of the herbal composition, the amount of folic acid is from 0.005 to about 0.2 wt. % of the herbal composition, the amount of vitamin A is from 0.02 to about 3 wt. % of the herbal composition, the amount of pyridoxine is from about 0.03 to about 10 wt. % of the herbal composition, the amount of ginger root or ginger root extract is from about 0.1 to about 20 wt. % of the herbal composition, the amount of glutathione is from about 0.05 to about 35 wt. % of the herbal composition, the amount of bromelain is from about 5 to about 75 wt. % of the herbal composition, the amount of selenium is from about 0.001 to about 0.2 wt. % of the herbal composition, and the amount of salicin is from about 1 to about 25 wt. % of the herbal composition.

According to another embodiment, an herbal composition comprises potassium, thiamin (vitamin B-1), magnesium, silymarin, zinc, folic acid and bromelain. These ingredients are commonly available and may be obtained from commercial sources. The herbal composition also may use any pharmaceutically acceptable salt of the above ingredients. In one specific embodiment, an herbal composition comprises an amount of potassium being from about 25 mg to 3,000 mg, an amount of thiamin being from about 1 mg to about 300 mg, an amount of magnesium being from about 25 mg to about 1,000 mg, an amount of silymarin being from about 25 mg to about 15,000 mg, an amount of zinc being from about 8 mg to about 400 mg, and an amount of folic acid being from about 0.2 mg to abut 1 mg, and an amount of bromelain being from about 150 mg to about 3,000 mg. It is desirable to have effective amounts of these ingredients in the herbal composition to treat, for example, hangovers.

In another specific embodiment using at least potassium, thiamin (vitamin B-1), magnesium, silymarin, zinc, folic acid and bromelain, the amount of potassium is from about 0.1 to about 98 wt. % of the herbal composition, the amount of thiamin is from about 0.01 to about 75 wt. % of the herbal composition, the amount of magnesium is from about 0.1 to about 95 wt. % of the herbal composition, the amount of silymarin is from about 0.5 to about 99.9 wt. % of the herbal composition, the amount of zinc is from about 0.05 to about 70 wt. % of the herbal composition, the amount of folic acid is from about 0.001 to about 0.5 wt. % of the herbal composition, and the amount of bromelain is from about 0.5 to about 95 wt. % of the herbal composition.

In another specific embodiment using at least potassium, thiamin, magnesium, silymarin, zinc, folic acid and bromelain, the amount of potassium is from about 2 to about 60 wt. % of the herbal composition, the amount of thiamin is from about 0.1 to about 25 wt. % of the herbal composition, the amount of magnesium is from about 4 to about 85 wt. % of the herbal composition, the amount of silymarin is from about 3 to about 75 wt. % of the herbal composition, the amount of zinc is from about 0.05 to about 50 wt. % of the herbal composition, the amount of folic acid is from about 0.1 to about 0.5 wt. % of the herbal composition, and the amount of bromelain is from about 0.5 to about 75 wt. % of the herbal composition.

According to another embodiment, the herbal composition may comprise additional ingredients besides potassium, thiamin, magnesium, silymarin, folic acid, zinc and bromelain. For example, the herbal composition may comprise potassium, thiamin, magnesium, silymarin, folic acid, zinc and bromelain and one or more of the following: ascorbic acid (vitamin C), riboflavin (vitamin B-2), proanthocyandins, and vitamin E. The herbal composition also may use any pharmaceutically acceptable salt of the above ingredients.

In another embodiment, an herbal composition comprises an amount of potassium being from about 25 mg to 3,000 mg, an amount of thiamin being from about 1 mg to about 300 mg, an amount of magnesium being from about 25 mg to about 1,000 mg, an amount of silymarin being from about 25 mg to about 15,000 mg, an amount of zinc being from about 8 mg to about 400 mg, an amount of folic acid being from about 0.2 mg to about 1 mg, an amount of bromelain being from about 150 mg to about 3,000 mg, an amount of ascorbic acid being from about 60 mg to about 2,500 mg, an amount of riboflavin being from about 1 mg to about 1,000 mg, and an amount of proanthocyandins being from about 40 mg to about 300 mg. It is desirable to have effective amounts of these ingredients in the herbal composition to treat, for example, hangovers.

In another specific embodiment, the amount of potassium is from about 0.1 to about 95 wt. % of the herbal composition, the amount of thiamin is from about 0.01 to about 60 wt. % of the herbal composition, the amount of magnesium is from about 0.1 to about 85 wt. % of the herbal composition, the amount of silymarin is from about 0.3 to about 99 wt. % of the herbal composition, the amount of zinc is from about 0.05 to about 70 wt. % of the herbal composition, the amount of folic acid is from 0.001 to about 0.5 wt. %, the amount of bromelain is from about 0.5 to about 95 wt. % of the herbal composition, the amount of ascorbic acid is from about 0.3 to about 95 wt. % of the herbal composition, the amount of riboflavin is from about 0.01 to about 85 wt. % of the herbal composition, and the amount of proanthocyandins is from about 0.1 to about 65 wt. % of the herbal composition.

In another specific embodiment, the amount of potassium is from about 1 to about 35 wt. % of the herbal composition, the amount of thiamin is from about 0.05 to about 10 wt. % of the herbal composition, the amount of magnesium is from about 1 to about 70 wt. % of the herbal composition, the amount of silymarin is from about 1 to about 60 wt. % of the herbal composition, the amount of zinc is from about 0.5 to about 50 wt. % of the herbal composition, the amount of folic acid is from about 0.1 to about 0.5 wt. % of the herbal composition, the amount of ascorbic acid is from about 3 to about 90 wt. % of the herbal composition, the amount of riboflavin is from about 0.05 to about 55 wt. % of the herbal composition, the amount of proanthocyandins is from about 2 to about 65 wt. % of the herbal composition and the amount of bromelain is from about 5 to about 75 wt. % of the herbal composition.

According to another embodiment, the herbal composition may comprise additional ingredients besides potassium, thiamin, magnesium, silymarin, folic acid, zinc and bromelain. For example, the herbal composition may comprise potassium, thiamin, magnesium, silymarin, folic acid, zinc and bromelain and one or more of the following: ascorbic acid (vitamin C), riboflavin, proanthocyandins, vitamin E, vitamin A, pyridoxine (vitamin B-6), ginger root or ginger root extract, glutathione, selenium and salicin. The herbal composition also may use any pharmaceutically acceptable salt of the above ingredients.

In another embodiment, an herbal composition comprises an amount of potassium being from about 25 mg to 3,000 mg, an amount of thiamin being from about 1 mg to about 300 mg, an amount of magnesium being from about 25 mg to about 1,000 mg, an amount of silymarin being from about 25 mg to about 15,000 mg, an amount of zinc being from about 10 mg to about 400 mg, an amount of folic acid being from about 0.2 mg to about 1 mg, an amount of bromelain being from about 150 mg to about 3,000 mg, an amount of ascorbic acid being from about 60 mg to about 2,500 mg, an amount of riboflavin being from about 1 mg to about 1,000 mg, an amount of proanthocyandins being from about 40 mg to about 300 mg, an amount of vitamin A being from about 1 mg to about 15 mg, an amount of pyridoxine being from about 1 mg to about 500 mg, an amount of ginger root being from about 5 mg to about 4,000 mg, an amount of glutathione being from about 2.5 mg to about 5,000 mg, an amount of selenium being from about 0.05 mg to about 0.7 mg, and an amount of salicin being from about 60 mg to about 120 mg. It is desirable to have effective amounts of these ingredients in the herbal composition to treat, for example, hangovers.

In a further specific embodiment, the amount of potassium is from about 0.1 to about 90 wt. % of the herbal composition, the amount of thiamin is from 0.001 to about 40 wt. % of the herbal composition, the amount of magnesium is from about 0.1 to about 70 wt. % of the herbal composition, the amount of silymarin is from about 0.1 to about 99 wt. % of the herbal composition, the amount of zinc is from about 0.02 to about 50 wt. % of the herbal composition, the amount of folic acid is from 0.001 to about 0.3 wt. % of the herbal composition, the amount of bromelain is from about 0.5 to about 95 wt. % of the herbal composition, the amount of ascorbic acid is from about 0.2 to about 90 wt. % of the herbal composition, the amount of riboflavin is from 0.001 to about 70 wt. % of the herbal composition, the amount of proanthocyandins is from about 0.1 to about 50 wt. % of the herbal composition, the amount of vitamin A is from 0.0001 to about 5 wt. % of the herbal composition, the amount of pyridoxine is from 0.001 to about 60 wt. % of the herbal composition, the amount of ginger root or ginger root extract is from about 0.01 to about 90 wt. % of the herbal composition, the amount of glutathione is from about 0.01 to about 95 wt. % of the herbal composition, the amount of selenium is from 0.0001 to about 0.2 wt. % of the herbal composition, and the amount of salicin is from about 0.15 to about 25 wt. % of the herbal composition.

In another specific embodiment, the amount of potassium is from about 1 to about 20 wt. % of the herbal composition, the amount of thiamin is from about 0.02 to about 5 wt. % of the herbal composition, the amount of magnesium is from about 10 to about 50 wt. % of the herbal composition, the amount of silymarin is from about 0.5 to about 40 wt. % of the herbal composition, the amount of zinc is from about 0.2 to about 35 wt. % of the herbal composition, the amount of folic acid is from 0.005 to about 0.2 wt. % of the herbal composition, the amount of bromelain is from about 5 to about 75 wt. % of the herbal composition, the amount of ascorbic acid is from about 2 to about 70 wt. % of the herbal composition, the amount of riboflavin is from about 0.02 to about 40 wt. % of the herbal composition, the amount of proanthocyandins is from about 1 to about 40 wt. % of the herbal composition, the amount of vitamin A is from 0.02 to about 3 wt. % of the herbal composition, the amount of pyridoxine is from about 0.03 to about 10 wt. % of the herbal composition, the amount of ginger root or ginger root extract is from about 0.1 to about 20 wt. % of the herbal composition, the amount of glutathione is from about 0.05 to about 35 wt. % of the herbal composition, the amount of selenium is from about 0.001 to about 0.2 wt. % of the herbal composition, and the amount of salicin is from about 1 to about 25 wt. % of the herbal composition.

In another embodiment, an herbal composition comprises potassium, thiamin, magnesium and silymarin. In one specific embodiment, an herbal composition comprises an amount of potassium being from about 25 mg to 3,000 mg, an amount of thiamin being from about 1 mg to about 300 mg, an amount of magnesium being from about 25 mg to about 1,000 mg, and an amount of silymarin being from about 25 mg to about 15,000 mg. It is desirable to have effective amounts of these ingredients in the herbal composition to treat, for example, hangovers.

In another specific embodiment using at least potassium, thiamin (vitamin B-1), magnesium and silymarin, the amount of potassium is from about 0.1 to about 98 wt. % of the herbal composition, the amount of thiamin is from about 0.01 to about 75 wt. % of the herbal composition, the amount of magnesium is from about 0.1 to about 95 wt. % of the herbal composition and the amount of silymarin is from about 0.5 to about 99.9 wt. % of the herbal composition.

In another specific embodiment using at least potassium, thiamin, magnesium and silymarin, the amount of potassium is from about 2 to about 60 wt. % of the herbal composition, the amount of thiamin is from about 0.1 to about 25 wt. % of the herbal composition, the amount of magnesium is from about 4 to about 85 wt. % of the herbal composition and the amount of silymarin is from about 3 to about 75 wt. % of the herbal composition.

In another specific embodiment using at least potassium, thiamin, magnesium and silymarin, the amount of potassium is from about 5 to about 55 wt. % of the herbal composition, the amount of thiamin is from about 0.1 to about 5 wt. % of the herbal composition, the amount of magnesium is from about 5 to about 75 wt. % of the herbal composition, and the amount of silymarin is from about 5 to about 75 wt. % of the herbal composition.

According to another embodiment, the herbal composition may comprise additional ingredients besides potassium, thiamin, magnesium and silymarin. For example, the herbal composition may comprise potassium, thiamin, magnesium, silymarin and N-acetyl-L-cysteine and one or more of the following: ascorbic acid (vitamin C), riboflavin (vitamin B-2), proanthocyandins, vitamin E, zinc, folic acid and bromelain. The herbal composition also may use any pharmaceutically acceptable salt of the above ingredients.

### Embodiment A

An herbal composition adapted to treat hangovers, the herbal composition comprising potassium, thiamin (vitamin B-1), magnesium, silymarin and N-acetyl-L-cysteine (acetylcysteine), or any pharmaceutically acceptable salts thereof.

### Embodiment B

The herbal composition of Embodiment A wherein potassium is from about 0.1 to about 98 wt. % of the herbal composition, thiamin is from about 0.01 to about 75 wt. % of the herbal composition, magnesium is from about 0.1 to about 95 wt. % of the herbal composition, silymarin is from about 0.5 to about 99.9 wt. % of the herbal composition, and N-acetyl-L-cysteine is from about 0.1 to about 90 wt. % of the herbal composition.

### Embodiment C

The herbal composition of Embodiment B wherein potassium is from about 2 to about 60 wt. % of the herbal composition, thiamin is from about 0.1 to about 25 wt. % of the herbal composition, magnesium is from about 4 to about 85 wt. % of the herbal composition, silymarin is from about 3 to about 75 wt. % of the herbal composition, and N-acetyl-L-cysteine is from about 3 to about 80 wt. % of the herbal composition.

### Embodiment D

The herbal composition of Embodiment C wherein potassium is from about 5 to about 55 wt. % of the herbal composition, thiamin is from about 0.1 to about 5 wt. % of the herbal composition, magnesium is from about 5 to about 75 wt. % of the herbal composition, silymarin is from about 5 to about 75 wt. % of the herbal composition, and N-acetyl-L-cysteine is from about 5 to about 75 wt. % of the herbal composition.

### Embodiment E

The herbal composition of Embodiment A wherein the herbal composition further comprises ascorbic acid (vitamin C) or any pharmaceutically acceptable salt thereof.

### Embodiment F

The herbal composition of Embodiment A wherein the herbal composition further comprises riboflavin or any pharmaceutically acceptable salt thereof.

### Embodiment G

The herbal composition of Embodiment A wherein the herbal composition further comprises proanthocyandins or any pharmaceutically acceptable salt thereof.

### Embodiment H

The herbal composition of Embodiment A wherein the herbal composition further comprises Vitamin E or any pharmaceutically acceptable salt thereof.

### Embodiment I

The herbal composition of Embodiment A wherein the herbal composition further comprises zinc or any pharmaceutically acceptable salt thereof.

### Embodiment J

The herbal composition of Embodiment A wherein the herbal composition further comprises folic acid or any pharmaceutically acceptable salt thereof.

### Embodiment K

The herbal composition of Embodiment A wherein the herbal composition further comprises ascorbic acid (vitamin C), riboflavin, proanthocyandins, zinc, folic acid, vitamin A, pyridoxine (vitamin B-6), ginger root or ginger root extract, glutathione (L-glutathione), bromelain, selenium and salicin or any pharmaceutically acceptable salt thereof.

### Embodiment L

The herbal composition of Embodiment A wherein the herbal composition further comprises ascorbic acid, riboflavin, proanthocyandins, zinc and folic acid, or any pharmaceutically acceptable salt thereof.

### Embodiment M

The herbal composition of Embodiment A wherein the herbal composition is a solution.

### Embodiment N

The herbal composition of Embodiment A wherein the herbal composition is a tablet or capsule.

### Process O

A method of treating hangovers, the method comprising the acts of: providing an herbal composition comprising potassium, thiamin (vitamin B-1), magnesium, silymarin and N-acetyl-L-cysteine (acetylcysteine) or any pharmaceutically acceptable salts thereof; and placing the herbal composition in the body so as to alleviate or reduce the effects of a hangover.

### Process P

The method of Process O wherein the placing of the herbal composition in the body is ingesting the herbal composition.

### Process Q

The method of Process O wherein potassium is from about 0.1 to about 98 wt. % of the herbal composition, thiamin is from about 0.01 to about 75 wt. % of the herbal composition, magnesium is from about 0.1 to about 95 wt. % of the herbal composition, silymarin is from about 0.5 to about 99.9 wt. % of the herbal composition, and N-acetyl-L-cysteine is from about 0.1 to about 90 wt. % of the herbal composition.

### Process R

The method of Process O wherein potassium is from about 2 to about 60 wt. % of the herbal composition, thiamin is from about 0.1 to about 25 wt. % of the herbal composition, magnesium is from about 4 to about 85 wt. % of the herbal composition, silymarin is from about 3 to about 75 wt. % of the herbal composition, and N-acetyl-L-cysteine is from about 3 to about 80 wt. % of the herbal composition.

### Process S

The method of Process R wherein potassium is from about 5 to about 55 wt. % of the herbal composition, thiamin is from about 0.1 to about 5 wt. % of the herbal composition, magnesium is from about 5 to about 75 wt. % of the herbal composition, silymarin is from about 5 to about 75 wt. % of the herbal composition, and N-acetyl-L-cysteine is from about 5 to about 75 wt. % of the herbal composition.

### Process T

The method of Process O wherein the herbal composition further comprises ascorbic acid (vitamin C) or any pharmaceutically acceptable salt thereof.

### Process U

The method of Process O wherein the herbal composition further comprises riboflavin or any pharmaceutically acceptable salt thereof.

### Process V

The method of Process O wherein the herbal composition further comprises proanthocyandins or any pharmaceutically acceptable salt thereof.

### Process W

The method of Process O wherein the herbal composition further comprises Vitamin E or any pharmaceutically acceptable salt thereof.

### Process X

The method of Process O wherein the herbal composition further comprises zinc or any pharmaceutically acceptable salt thereof.

### Process Y

The method of Process O wherein the herbal composition further comprises folic acid or any pharmaceutically acceptable salt thereof.

### Process Z

The method of Process O wherein the herbal composition further comprises ascorbic acid (vitamin C), riboflavin, proanthocyandins, zinc, folic acid, vitamin A, pyridoxine (vitamin B-6), ginger root or ginger root extract, glutathione (L-glutathione), bromelain, selenium and salicin or any pharmaceutically acceptable salt thereof.

### Process AA

The method of Process O wherein the herbal composition further comprises ascorbic acid, riboflavin, proanthocyandins, zinc and folic acid, or any pharmaceutically acceptable salts thereof.

### Process BB

The method of Process O wherein the herbal composition is a solution.

### Process CC

The method of Process O wherein the herbal composition is a tablet or capsule.

### Embodiment DD

An herbal composition adapted to treat hangovers, the herbal composition comprising potassium, thiamin (vitamin B-1), magnesium, silymarin, folic acid, zinc and bromelain or any pharmaceutically acceptable salts thereof.

While the present invention has been described with reference to one or more particular embodiments, those skilled in the art will recognize that many changes may be made thereto without departing from the spirit and scope of the present invention. Each of these embodiments, and obvious variations thereof, is contemplated as falling within the spirit and scope of the invention as defined by the appended claims.

What is claimed is:

1. An herbal composition adapted to treat hangovers, the herbal composition comprising potassium, thiamin (vitamin B-1), magnesium, silymarin and N-acetyl-L-cysteine (acetylcysteine), or any pharmaceutically acceptable salts thereof.

2. The herbal composition of claim 1 wherein potassium is from about 2 to about 60 wt. % of the herbal composition, thiamin is from about 0.1 to about 25 wt. % of the herbal composition, magnesium is from about 4 to about 85 wt. % of the herbal composition, silymarin is from about 3 to about 75 wt. % of the herbal composition, and N-acetyl-L-cysteine is from about 3 to about 80 wt. % of the herbal composition.

3. The herbal composition of claim 2 wherein potassium is from about 5 to about 55 wt. % of the herbal composition, thiamin is from about 0.1 to about 5 wt. % of the herbal composition, magnesium is from about 5 to about 75 wt. % of the herbal composition, silymarin is from about 5 to about 75 wt. % of the herbal composition, and N-acetyl-L-cysteine is from about 5 to about 75 wt. % of the herbal composition.

4. The herbal composition of claim 1 wherein the herbal composition further comprises ascorbic acid (vitamin C), riboflavin, proanthocyandins, zinc, folic acid, vitamin A, pyridoxine (vitamin B-6), ginger root or ginger root extract, glutathione (L-glutathione), bromelain, selenium and salicin or any pharmaceutically acceptable salt thereof.

5. The herbal composition of claim 1 wherein the herbal composition further comprises ascorbic acid, riboflavin, proanthocyandins, zinc and folic acid, or any pharmaceutically acceptable salt thereof.

6. The herbal composition of claim 1 wherein the herbal composition is a tablet or capsule.

7. A method of treating hangovers, the method comprising the acts of providing an herbal composition comprising potassium, thiamin (vitamin B-1), magnesium, silymarin and N-acetyl-L-cysteine (acetylcysteine) or any pharmaceutically acceptable salts thereof; and placing the herbal composition in the body so as to alleviate or reduce the effects of a hangover.

8. The method of claim 7 wherein the placing of the herbal composition in the body is ingesting the herbal composition.

9. The method of claim 7 wherein potassium is from about 2 to about 60 wt. % of the herbal composition, thiamin is from about 0.1 to about 25 wt. % of the herbal composition, magnesium is from about 4 to about 85 wt. % of the herbal composition, silymarin is from about 3 to about 75 wt. % of the herbal composition, and N-acetyl-L-cysteine is from about 3 to about 80 wt. % of the herbal composition.

10. The method of claim 7 wherein potassium is from about 5 to about 55 wt. % of the herbal composition, thiamin is from about 0.1 to about 5 wt. % of the herbal composition, magnesium is from about 5 to about 75 wt. % of the herbal composition, silymarin is from about 5 to about 75 wt. % of the herbal composition, and N-acetyl-L-cysteine is from about 5 to about 75 wt. % of the herbal composition.

11. The method of claim 7 wherein the herbal composition further comprises ascorbic acid (vitamin C), riboflavin, proanthocyandins, zinc, folic acid, vitamin A, pyridoxine (vitamin B-6), ginger root or ginger root extract, glutathione (L-glutathione), bromelain, selenium and salicin or any pharmaceutically acceptable salt thereof.

12. The method of claim 7 wherein the herbal composition further comprises ascorbic acid, riboflavin, proanthocyandins, zinc and folic acid, or any pharmaceutically acceptable salts thereof.

13. The method of claim 7 wherein the herbal composition is a solution.

14. The method of claim 7 wherein the herbal composition is a tablet or capsule.

15. An herbal composition adapted to treat hangovers, the herbal composition comprising potassium, thiamin (vitamin B-1), magnesium, silymarin, folic acid, zinc and bromelain or any pharmaceutically acceptable salts thereof.

16. The herbal composition of claim 15 wherein potassium is from about 2 to about 60 wt. % of the herbal composition, thiamin is from about 0.1 to about 25 wt. % of the herbal composition, magnesium is from about 4 to about 85 wt. % of the herbal composition, silymarin is from about 3 to about 75 wt. % of the herbal composition, folic acid is from about 0.1 to about 0.5 wt. % of the herbal composition, zinc is from about 0.5 to about 50 wt. % of the herbal composition, bromelain is from about 0.5 to about 75 wt. % of the herbal composition.

17. The herbal composition of claim 15 wherein the herbal composition further comprises ascorbic acid, riboflavin and proanthocyandins, or any pharmaceutically acceptable salt thereof.

18. The herbal composition of claim 15 wherein the herbal composition is a tablet or capsule.

19. The herbal composition of claim 15 wherein the herbal composition is a solution.

20. A method of treating hangovers, the method comprising the acts of providing an herbal composition comprising potassium, thiamin (vitamin B-1), magnesium, and silymarin or any pharmaceutically acceptable salts thereof; and placing the herbal composition in the body so as to alleviate or reduce the effects of a hangover.

21. The method of claim 20 wherein potassium is from about 2 to about 60 wt. % of the herbal composition, thiamin is from about 0.1 to about 25 wt. % of the herbal composition, magnesium is from about 4 to about 85 wt. % of the herbal composition, and silymarin is from about 3 to about 75 wt. % of the herbal composition.

22. The method of claim 20 wherein potassium is from about 5 to about 55 wt. % of the herbal composition, thiamin is from about 0.1 to about 5 wt. % of the herbal composition, magnesium is from about 5 to about 75 wt. % of the herbal composition, and silymarin is from about 5 to about 75 wt. % of the herbal composition.

23. The method of claim 20 wherein the herbal composition further includes folic acid, zinc and bromelain or any pharmaceutically acceptable salts thereof.
